(19) United States
(12) Patent Application Publication
(10) Pub. No.: US 2025/0166416 A1
(43) Pub. Date: May 22, 2025

(54) SYSTEM AND METHOD FOR HAND MOVEMENTS RECOGNITION AND ANALYSIS

(71) Applicant: METATECH (AP) INC., New Taipei City (TW)

(72) Inventors: TSUNG-CHI CHEN, New Taipei City (TW); Ying-chen Yang, Taipei City (TW); Yeong-Sung Lin, Taipei City (TW)

(21) Appl. No.: 18/952,699

(22) Filed: Nov. 19, 2024

Related U.S. Application Data
(60) Provisional application No. 63/601,235, filed on Nov. 21, 2023.

(51) Int. Cl. G06V 40/20; G06T 7/70; G06V 10/82; G06V 40/10; G08B 3/10

(52) U.S. Cl. CPC G06V 40/28; G06T 7/70; G06V 10/82; G06V 40/10; G08B 3/10; G06T 2207/20081; G06T 2207/20084; G06T 2207/30196

(57) ABSTRACT

A system for hand movements recognition and analysis comprises an image capture module, a recognition module, and a movement analysis module. The image capture module is installed in an operational area and is configured to capture a continuous image of the at least one hand movement of an operator during an operation period. The recognition module is coupled with the image capture module and comprises a 2D joint recognition model, a joint 3D coordinate recognition model and a hand skeletal joint position global optimization model to recognize a hand joint position of the operator based on the continuous image and generate a hand joint position coordinate. The movement analysis module comprises a hand movement analysis model for receiving the hand joint position coordinate, analyzing and comparing the hand joint position coordinate of the operator with a standard hand movement parameter set, and generating an analysis and comparison result to evaluate the correctness of the at least one hand movement of the operator in the operating area during the operation period.

### BACKGROUND OF THE INVENTION

### 1. Field of the invention

The present invention relates to a system and method for hand movements recognition and analysis, particularly one involving machine learning.

### 2. Description of the Prior Art

Cell therapy is a method that utilizes biology, cytology, and molecular biology to repair, replace, or improve the function of damaged or failing tissues and organs. In recent years, cell therapy development has proliferated due to the approval of the Regulations Governing the Application or Use of Specific Medical Techniques or Examinations, and Medical Devices (commonly referred to as the “Special Regulations”). Cell therapy primarily involves administering processed human cells into a patient's body to promote the growth of necessary cells (e.g., knee cartilage) or target diseased cells (e.g., cancer cells). In detail, the common cell therapy is to extract the cells from the patient's body, activate and increase the value of the cells in a sterile and dust-free operation space, and further produce cell therapy products to be injected back into the patient's body in order to achieve therapeutic and restorative effects.

In the manufacturing process of cell therapy products, the operating environment must maintain a high level of cleanliness, and all equipment and workstations must be sterile to prevent contamination that could compromise the effectiveness of the therapy. The operator's hand movements, operating sequence, and habits are closely linked to the quality of the cell therapy product. For example, due to habitual practices, an operator may frequently move their hands over the culture dishes, potentially causing bacteria from their hands to fall into the dishes. Similarly, improper operation, such as overly rapid pipetting of the culture medium, could cause the culture fluid to splash into the pipette, introducing dust or contaminants from the pipette into the cell culture dish. These factors can result in contamination during the production process. Additionally, in subculture processes, if the cell collection steps are not performed accurately (such as accidentally extracting cells while removing old culture medium or inaccurately measuring the volume of collected cells) may be inconsistencies in cell density or confluence across batches, affecting the overall quality and causing batch variations.

Operators may also need to be able to detect operational abnormalities due to the continuous nature of their movements or habitual practices. Furthermore, contamination caused by microorganisms and dust particles in the environment cannot be detected visually. As a result, contamination often goes unnoticed until the final harvest, which could take several days, revealing product contamination only at a late stage. In summary, the correctness of hand movements, operating sequence, and habits during cell therapy processes directly affects the final product's quality. However, current technologies in the market lack hand recognition systems designed to ensure the correctness of operations specifically for cell therapy applications.

While conventional technologies offer hand recognition systems that analyze and evaluate the correctness of hand movements based on joint points in a single image, the systems often face challenges during real-world operations. Specifically, overlapping hands or blind spots in the recognition system may cause distortions in the measured distances between hand joints, leading to inaccurate analysis results. Moreover, when the conventional hand recognition system experiences a distortion in joint-point measurements from a single image, the detection accuracy further deteriorates when applied to continuous image sequences, resulting in even more significant inaccuracies.

Therefore, it is necessary to develop a new system and method for hand movements recognition and analysis to address the shortcomings of the conventional systems mentioned above.

### SUMMARY OF THE INVENTION

In view of this, one scope of the present invention is to provide a system for hand movements recognition and analysis for evaluating the correctness of at least one hand movement performed by an operator in an operational area during an operation period. The system for hand movements recognition and analysis comprises an image capture module, a recognition module, and a movement analysis module. The image capture module is installed in the operational area, and is configured to capture a continuous image of the at least one hand movement of the operator during the operation period. The recognition module is coupled with the image capture module. The recognition module comprises a 2D joint recognition model, a joint 3D coordinate recognition model and a hand skeletal joint position global optimization model. The 2D joint recognition model is configured to recognize a hand joint position coordinate of the operator based on the continuous image. The joint 3D coordinate recognition model is coupled with the 2D joint recognition model, and configured to generate a hand joint position coordinate of the operator based on a plurality of continuous images from various perspectives captured by a plurality of image capture modules in the operational area during the operation period, wherein the hand joint position coordinate is a 3D coordinate. The hand skeletal joint position global optimization model is coupled with the joint 3D coordinate recognition model, and configured to optimize the hand joint position coordinate based on a finger bone length constraint and a finger bone joint constraint of the operator through a global optimization processing to improve the accuracy of the hand joint position coordinate. The 2D joint recognition model, the joint 3D coordinate recognition model, and the hand skeletal joint position global optimization model are trained on a hand movement dataset by a first machine learning method. The movement analysis module is coupled with the recognition module. The movement analysis module comprises a hand movement analysis model configured to receive the hand joint position coordinate, analyze and compare the hand joint position coordinate of the operator with a standard hand movement parameter set, and generate an analysis and comparison result to evaluate the correctness of the at least one hand movement performed by the operator during the operation period, wherein the hand movement analysis model is trained on a standard operational hand movement dataset by a second machine learning method.

Wherein, the 3D coordinate is calculated by triangulating an intersection of a ray for each of the image capture modules, or by using a midpoint calculation of the shortest distance of each ray from the image capture modules as an approximate intersection point.

Wherein, the system for hand movements recognition and analysis further comprises a movement classification recognition model, configured to recognize and classify different types of hand movements based on the hand joint position coordinate in the continuous images captured by the image capture module through a third machine learning method.

Wherein, the system for hand movements recognition and analysis further comprises a movement and sequence accuracy recognition model, configured to analyze and compare the hand joint position coordinate with the standard hand movement parameter set, and determine whether the at least one hand movement of the operator and an operation sequence are correct.

Wherein, the system for hand movements recognition and analysis further comprises a notification module, coupled with the movement analysis module, the notification module configured to send a notification message to alert the operator based on the analysis and comparison result generated by analyzing and comparing the hand joint position coordinate of the operator in the operational area during the operation period with the standard hand movement parameter set.

Wherein, the notification message further comprises an audio notification and a light signal notification.

Wherein, if the analysis and comparison result between the hand joint position coordinate of the operator in the operational area during the operation period and the standard hand movement parameter set does not match, the notification module generates the audio notification, wherein the audio notification is an error alert warning.

Wherein, the image capture module comprises a camera installed in the operational area.

Wherein, the operational area comprises an experimental operation platform, a sterile operation platform, and a cell handling station.

Wherein, the first machine learning method, the second machine learning method and the third machine learning method further comprise at least one of the following: an Artificial Neural Network (ANN), a Convolutional Neural Network (CNN), a Recurrent Neural Network (RNN), a Decision Tree, a Support Vector Machine (SVM), a Random Forest, a K-Nearest Neighbors (KNN) algorithm, K-Means Clustering, Principal Component Analysis (PCA), Linear Regression, Logistic Regression, Gradient Boosting Machines, a Deep Belief Network (DBN), a Recursive Neural Network (RecNN), Reinforcement Learning, an Autoencoder, Gaussian Processes, and a Complex Neural Network.

Another scope of the present invention is to provide a method for hand movements recognition and analysis. The method comprises the following steps: an image capture module capturing a continuous image of the at least one hand movement of the operator during the operation period; a 2D joint recognition model of a recognition module recognizing a hand joint position of the operator based on the continuous image; a joint 3D coordinate recognition model of the recognition module generating a hand joint position coordinate of the operator based on a plurality of continuous images from various perspectives captured by a plurality of image capture modules in the operational area during the operation period, wherein the hand joint position coordinate is a 3D coordinate; a hand skeletal joint position global optimization model of the recognition module optimizing the hand joint position coordinate based on a finger bone length constraint and a finger bone joint constraint of the operator through a global optimization processing to improve the accuracy of the hand joint position coordinate, wherein the 2D joint recognition model, the joint 3D coordinate recognition model, and the hand skeletal joint position global optimization model are trained on a hand movement dataset by a first machine learning method; and a hand movement analysis model of a movement analysis module receiving the hand joint position coordinate, analyzing and comparing the hand joint position coordinate of the operator with a standard hand movement parameter set, and generating an analysis and comparison result to evaluate the correctness of the at least one hand movement performed by the operator during the operation period, wherein the hand movement analysis model is trained on a standard operational hand movement dataset by a second machine learning method.

Wherein, the 3D coordinate is calculated by triangulating an intersection of a ray for each of the image capture modules, or by using a midpoint calculation of the shortest distance of each ray from the image capture modules as an approximate intersection point.

Wherein, the method for hand movements recognition and analysis further comprises the following steps: a movement classification recognition model of the movement analysis module recognizing and classifying different types of hand movements based on the hand joint position coordinate in the continuous images captured by the image capture module through a third machine learning method.

Wherein, the method for hand movements recognition and analysis further comprises the following steps: a movement and sequence accuracy recognition model of the movement analysis module analyzing and comparing the hand joint position coordinate with the standard hand movement parameter set, and determining whether the at least one hand movement of the operator and an operation sequence are correct.

Wherein, the method for hand movements recognition and analysis further comprises the following steps: a notification module sending a notification message to alert the operator based on the analysis and comparison result generated by analyzing and comparing the hand joint position coordinate of the operator in the operational area during the operation period with the standard hand movement parameter set.

Wherein, the notification message further comprises an audio notification and a light signal notification.

Wherein, if the analysis and comparison result between the hand joint position coordinate of the operator in the operational area during the operation period and the standard hand movement parameter set does not match, the notification module generates the audio notification, wherein the audio notification is an error alert warning.

Wherein, the image capture module comprises a camera installed in the operational area.

Wherein, the operational area comprises an experimental operation platform, a sterile operation platform, and a cell handling station.

Wherein, the first machine learning method, the second machine learning method and the third machine learning method further comprise at least one of the following: an Artificial Neural Network (ANN), a Convolutional Neural Network (CNN), a Recurrent Neural Network (RNN), a Decision Tree, a Support Vector Machine (SVM), a Random Forest, a K-Nearest Neighbors (KNN) algorithm, K-Means Clustering, Principal Component Analysis (PCA), Linear Regression, Logistic Regression, Gradient Boosting Machines, a Deep Belief Network (DBN), a Recursive Neural Network (RecNN), Reinforcement Learning, an Autoencoder, Gaussian Processes, and a Complex Neural Network.

In summary, the invention provides a system and method for hand movements recognition and analysis that integrates artificial intelligence with a hand recognition system to analyze the correctness of an operator's hand movements. This approach enhances both the quality and consistency of process outcomes. Utilizing the hand skeletal joint position global optimization model, the system performs thorough optimization based on the operator's finger bone lengths and joint movement constraints. This improves the accuracy of hand joint position recognition while reducing distortion during the recognition process. Additionally, through the notification module, the system issues alerts based on the analysis and comparison result, the system informs the operator of any incorrect movements, enabling real-time correction and thereby improving the accuracy of hand movements. Wherein, the analysis and comparison result is generating by analyzing and comparing the hand joint position coordinate of the operator in the operational area during the operation period with the standard hand movement parameter set.

### BRIEF DESCRIPTION OF THE APPENDED DRAWINGS

FIG. 1 is a functional block diagram of a system for hand movements recognition and analysis according to an embodiment of the present invention.

FIG. 2 is a functional block diagram of a system for hand movements recognition and analysis according to another embodiment of the present invention.

FIG. 3 is a flowchart diagram of a method for hand movements recognition and analysis according to an embodiment of the present invention.

FIG. 4 is a flowchart diagram of a method for hand movements recognition and analysis according to another embodiment of the present invention.

FIG. 5 is a functional block diagram of the movement analysis module according to FIG. 1.

### DETAILED DESCRIPTION OF THE INVENTION

For the sake of the advantages, spirits and features of the present invention can be understood more easily and clearly, the detailed descriptions and discussions will be made later by way of the embodiments and with reference of the diagrams. It is worth noting that these embodiments are merely representative embodiments of the present invention, wherein the specific methods, devices, conditions, materials and the like are not limited to the embodiments of the present invention or corresponding embodiments. Moreover, the devices in the figures are only used to express their corresponding positions and are not drawing according to their actual proportion.

In the description of this specification, the description with reference to the terms “an embodiment”, “another embodiment” or “part of an embodiment” means that a particular feature, structure, material or characteristic described in connection with the embodiment including in at least one embodiment of the present invention. In this specification, the schematic representations of the above terms do not necessarily refer to the same embodiment. Furthermore, the particular features, structures, materials or characteristics described may be combined in any suitable manner in one or more embodiments. Furthermore, the indefinite articles “a” and “an” preceding a device or element of the present invention are not limiting on the quantitative requirement (the number of occurrences) of the device or element. Thus, “a” should be read to include one or at least one, and a device or element in the singular also includes the plural unless the number clearly refers to the singular.

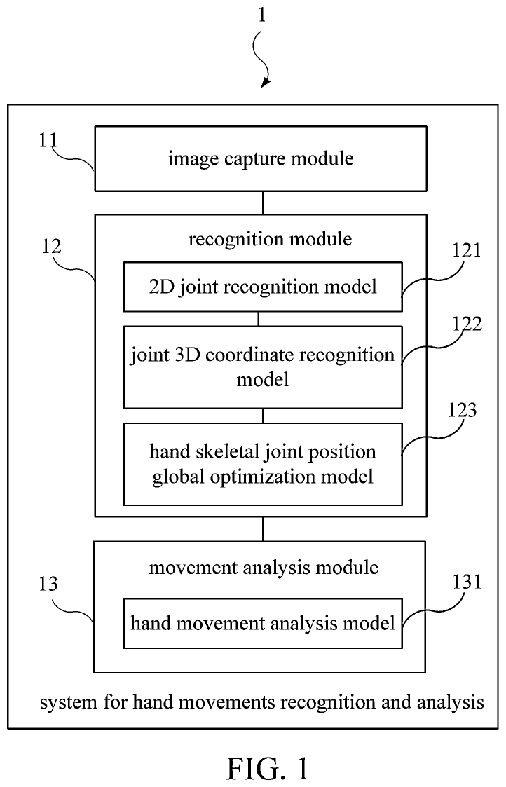

Please refer to FIG. 1. FIG. 1 is a functional block diagram of a system 1 for hand movements recognition and analysis according to an embodiment of the present invention. This embodiment of the system 1 for hand movements recognition and analysis can be used to evaluate the correctness of at least one hand movement performed by an operator in an operational area during an operation period. The system 1 for hand movements recognition and analysis comprises an image capture module 11, a recognition module 12, and a movement analysis module 13. The image capture module 11 is installed in the operational area, and is configured to capture a continuous image of the at least one hand movement of the operator during the operation period. The recognition module 12 is coupled with the image capture module 11. The recognition module 12 comprises a 2D joint recognition model 121, a joint 3D coordinate recognition model 122 and a hand skeletal joint position global optimization model 123. The 2D joint recognition model 121 is configured to recognize a hand joint position coordinate of the operator based on the continuous image. The joint 3D coordinate recognition model 122 is coupled with the 2D joint recognition model 121, and configured to generate a hand joint position coordinate of the operator based on a plurality of continuous images from various perspectives captured by a plurality of image capture modules 11 in the operational area during the operation period, wherein the hand joint position coordinate is a 3D coordinate. The hand skeletal joint position global optimization model 123 is coupled with the joint 3D coordinate recognition model 122, and configured to optimize the hand joint position coordinate based on a finger bone length constraint and a finger bone joint constraint of the operator through a global optimization processing to improve the accuracy of the hand joint position coordinate and reduce distortion during the recognition process. The 2D joint recognition model 121, the joint 3D coordinate recognition model 122, and the hand skeletal joint position global optimization model 123 are trained on a hand movement dataset by a first machine learning method. The movement analysis module 13 is coupled with the recognition module 12. The movement analysis module 13 comprises a hand movement analysis model 131 configured to receive the hand joint position coordinate, analyze and compare the hand joint position coordinate of the operator with a standard hand movement parameter set, and generate an analysis and comparison result to evaluate the correctness of the at least one hand movement performed by the operator during the operation period, wherein the hand movement analysis model 131 is trained on a standard operational hand movement dataset by a second machine learning method.

In this embodiment, the 3D coordinate is calculated by triangulating an intersection of a ray for each of the image capture modules 11, or by using a midpoint calculation of the shortest distance of each ray from the image capture modules 11 as an approximate intersection point. Additionally, the standard hand movement parameter set can be defined based on the hand movements associated with the batch that achieved the highest final product quality. This batch serves as the basis for determining the standard operational procedures. However, in practice, this parameter set is more comprehensive than this approach, and users can customize the parameters based on their specific requirements or operational needs. In this embodiment, the operational hand movement dataset can contain the hand movement parameters of correct hand movements performed by different operators during various operational processes. In practice, operators may vary palm size, hand width, and distances between joints, though their overall hand structures are similar. Therefore, applying machine learning methods to the operational hand movement dataset makes it possible to derive the optimal operational hand movement parameters for each operator, which form the standard hand movement parameter set.

In other embodiments, the image capture module 11 can include a camera installed in the operational area. In practice, the operational area can comprise an experimental operation platform, a sterile operation platform, a cell handling station, or other platforms or locations where process operations are conducted. The joint 3D coordinate recognition model 122 captures hand images of the operator from multiple perspectives using several cameras. It reconstructs the 3D coordinates of the joints by calculating the intersections of rays from each camera through triangulation. If precise intersections cannot be achieved, the midpoint method is used to calculate the midpoint of the shortest distance between the rays as an approximate intersection point, providing accurate and stable 3D positioning. Additionally, the hand skeletal joint position global optimization model 123 refines the joint position recognition accuracy by performing global optimization based on the operator's finger bone lengths and joint movement constraints, thereby reducing distortion during the recognition process. The optimization process involves extracting 2D key points from multi-perspective images, converting them into a 3D model using triangulation, and then performing comprehensive corrections based on finger bone lengths and joint constraints. The model posture is further adjusted and projected back onto the original images using pose control and projection functions. Through these processes, the model generates an optimized 3D hand skeletal model. This module not only improves the accuracy of joint position recognition but also minimizes distortion during the recognition process, ensuring the model's stability and precision.

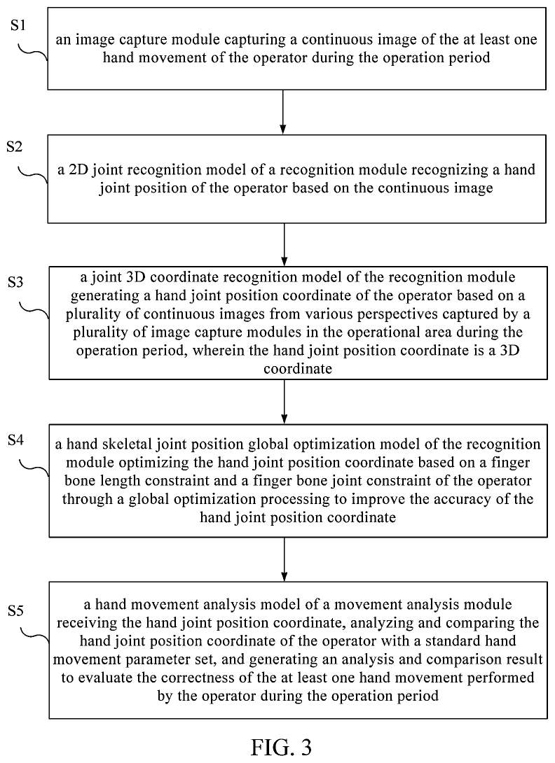

Please refer to FIG. 1 and FIG. 3. FIG. 3 is a flowchart diagram of a method for hand movements recognition and analysis according to an embodiment of the present invention. The steps in the process shown in FIG. 3 can be implemented using the system 1 for hand movements recognition and analysis described in FIG. 1. As shown in FIG. 3, the method comprises the following steps: step S1: an image capture module 11 capturing a continuous image of the at least one hand movement of the operator during the operation period; step S2: a 2D joint recognition model 121 of a recognition module 12 recognizing a hand joint position of the operator based on the continuous image; step S3: a joint 3D coordinate recognition model 122 of the recognition module 12 generating a hand joint position coordinate of the operator based on a plurality of continuous images from various perspectives captured by a plurality of image capture modules 11 in the operational area during the operation period, wherein the hand joint position coordinate is a 3D coordinate; step S4: a hand skeletal joint position global optimization model 123 of the recognition module 12 optimizing the hand joint position coordinate based on a finger bone length constraint and a finger bone joint constraint of the operator through a global optimization processing to improve the accuracy of the hand joint position coordinate; and step S5: a hand movement analysis model 131 of a movement analysis module 13 receiving the hand joint position coordinate, analyzing and comparing the hand joint position coordinate of the operator with a standard hand movement parameter set, and generating an analysis and comparison result to evaluate the correctness of the at least one hand movement performed by the operator during the operation period. Wherein the 2D joint recognition model 121, the joint 3D coordinate recognition model 122, and the hand skeletal joint position global optimization model 123 are trained on a hand movement dataset by a first machine learning method; the hand movement analysis model 131 is trained on a standard operational hand movement dataset by a second machine learning method.

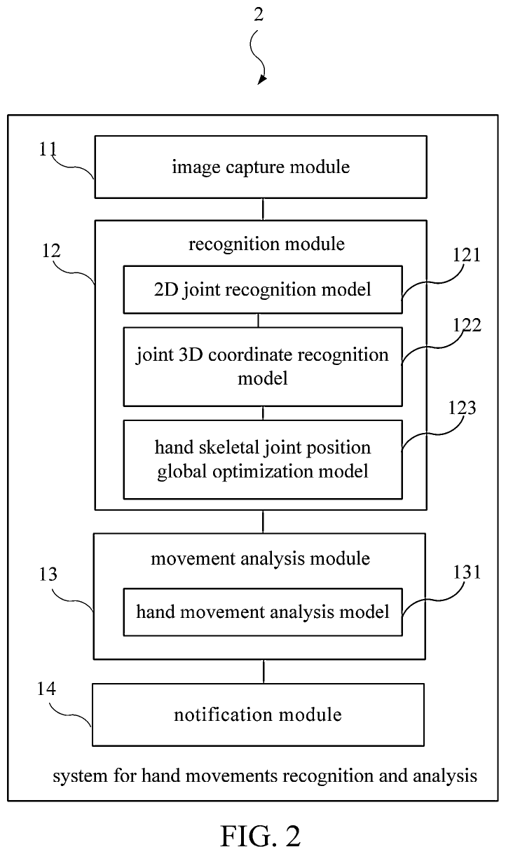

Please refer to FIG. 2. FIG. 2 is a functional block diagram of a system 2 for hand movements recognition and analysis according to another embodiment of the present invention. The present embodiment differs from the above embodiment in that the system 2 for hand movements recognition and analysis of the present embodiment further comprises a notification module 14. The notification module 14 is coupled with the movement analysis module 13. The notification module 14 is configured to send a notification message to alert the operator based on the analysis and comparison result generated by analyzing and comparing the hand joint position coordinate of the operator in the operational area during the operation period with a standard hand movement parameter set. In this embodiment, the notification message further comprises an audio notification and a light signal notification. If the analysis and comparison result between the hand joint position coordinate of the operator in the operational area during the operation period and the standard hand movement parameter set does not match, the notification module 14 generates the audio notification, wherein the audio notification is an error alert warning. Conversely, if the analysis and comparison result is a match, the notification module 14 will not issue the audio notification. Additionally, a light signal can be used to indicate status: a green light shows that the analysis and comparison result is a match, while a red light indicates that the analysis and comparison result does not match. However, it is not limited to this in practice. Users can also set the notification message according to their own needs or the operation process.

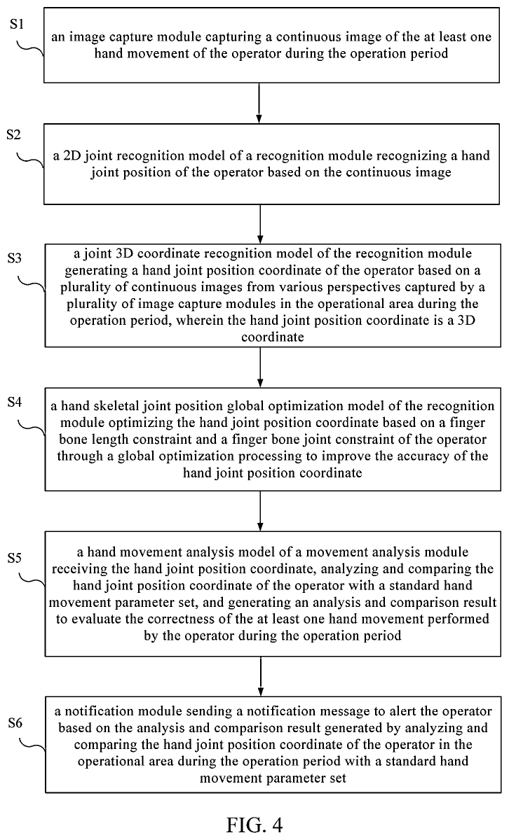

Please refer to FIG. 2 and FIG. 4. FIG. 4 is a flowchart diagram of a method for hand movements recognition and analysis according to another embodiment of the present invention. The steps in the method shown in FIG. 4 can be implemented using the system 2 for hand movements recognition and analysis described in FIG. 2. As shown in FIG. 4, in this embodiment, the method for hand movements recognition and analysis further comprises step S6, which follows step S5: step S6: a notification module 14 sending a notification message to alert the operator based on the analysis and comparison result generated by analyzing and comparing the hand joint position coordinate of the operator in the operational area during the operation period with a standard hand movement parameter set. Please note that the other modules, models, and their corresponding functionalities in this embodiment of system 2 are generally the same as those described in the previous embodiment. Therefore, their detailed descriptions are omitted here for brevity.

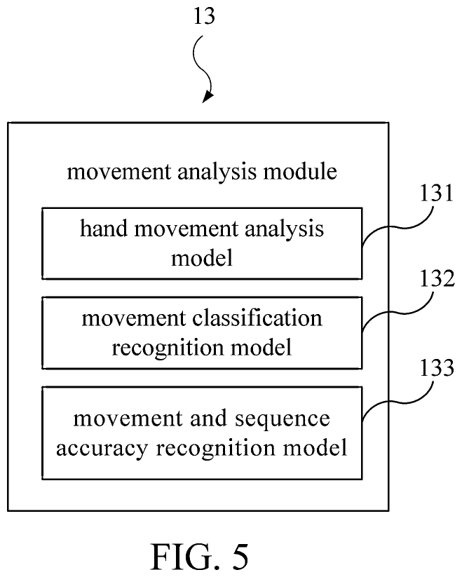

In another embodiment, the movement analysis module 13 can further include additional models. Please refer to FIG. 5. FIG. 5 is a functional block diagram of the movement analysis module 13 according to FIG. 1. As shown in FIG. 5, the movement analysis module 13 can further comprise a movement classification recognition model 132 and a movement and sequence accuracy recognition model 133, both of which use AI technology to enhance the accuracy of hand motion recognition and the judgment of operational correctness. The movement classification recognition model 132 is configured to recognize and classify different types of hand movements based on the hand joint position coordinate in the continuous images captured by the image capture module 11 through a third machine learning method. Using machine learning methods, the movement classification recognition model 132 can recognize and classify different types of hand movements, such as gripping, rotating, or grasping, based on a trained hand motion dataset, and quickly and accurately provide real-time motion classification results. The movement and sequence accuracy recognition model 133 focuses on the accuracy of the motion flow. The movement and sequence accuracy recognition model 133 is configured to analyze and compare the hand joint position coordinate with the standard hand movement parameter set, and determine whether the at least one hand movement of the operator and an operation sequence are correct. It compares the operator's hand joint position coordinates with a preset standard operational hand movement dataset. This comparison can detect whether the operator completes the designated motions in the correct sequence and assess the accuracy of the operation. By analyzing the analysis and comparison result, the movement and sequence accuracy recognition model 133 can evaluate whether the operator's hand movements and process during operation meet the standard, ensuring the accuracy and consistency of the operation. Furthermore, in this embodiment, the method for hand movements recognition and analysis can be combined with the movement classification recognition model 132 and the movement and sequence accuracy recognition model 133, further including the following steps: a movement classification recognition model 132 of the movement analysis module 13 recognizing and classifying different types of hand movements based on the hand joint position coordinate in the continuous images captured by the image capture module 11 through a third machine learning method; and a movement and sequence accuracy recognition model 133 of the movement analysis module 13 analyzing and comparing the hand joint position coordinate with the standard hand movement parameter set, and determining whether the at least one hand movement of the operator and an operation sequence are correct.

In the system for hand movements recognition and analysis of the present embodiment, the first machine learning method, the second machine learning method and the third machine learning method can further comprise any of the following: Artificial Neural Networks (ANNs), Convolutional Neural Networks (CNNs), Recurrent Neural Networks (RNNs), Decision Trees, Support Vector Machines (SVM), Random Forests, K-Nearest Neighbors (KNN), K-Means Clustering, Principal Component Analysis (PCA), Linear Regression, Logistic Regression, Gradient Boosting Machines, Deep Belief Networks (DBN), Recursive Neural Networks (RecNN), Reinforcement Learning, Autoencoders, Gaussian Processes, Complex Neural Networks, or any other machine learning or neural network algorithms. These methods are applied to train the datasets based on user requirements, allowing for selecting suitable machine learning or neural network algorithms according to the user's needs.

In summary, the invention provides a system and method for hand movements recognition and analysis that integrates artificial intelligence with a hand recognition system to analyze the correctness of an operator's hand movements. This approach enhances both the quality and consistency of process outcomes. Utilizing the hand skeletal joint position global optimization model, the system performs thorough optimization based on the operator's finger bone lengths and joint movement constraints. This improves the accuracy of hand joint position recognition while reducing distortion during the recognition process. Additionally, through the notification module, the system issues alerts based on the analysis and comparison result, the system informs the operator of any incorrect movements, enabling real-time correction and thereby improving the accuracy of hand movements. Wherein, the analysis and comparison result is generating by analyzing and comparing the hand joint position coordinate of the operator in the operational area during the operation period with the standard hand movement parameter set.

With the examples and explanations mentioned above, the features and spirits of the invention are hopefully well described. More importantly, the present invention is not limited to the embodiment described herein. Those skilled in the art will readily observe that numerous modifications and alterations of the device may be made while retaining the teachings of the invention. Accordingly, the above disclosure should be construed as limited only by the metes and bounds of the appended claims.

What is claimed is:

1. A system for hand movements recognition and analysis for evaluating the correctness of at least one hand movement performed by an operator in an operational area during an operation period, the system comprising:
an image capture module, installed in the operational area, configured to capture a continuous image of the at least one hand movement of the operator during the operation period;
a recognition module, coupled with the image capture module, the recognition module comprising:
a 2D joint recognition model, configured to recognize a hand joint position of the operator based on the continuous image;
a joint 3D coordinate recognition model, coupled with the 2D joint recognition model, and configured to generate a hand joint position coordinate of the operator based on a plurality of continuous images from various perspectives captured by a plurality of image capture modules in the operational area during the operation period, wherein the hand joint position coordinate is a 3D coordinate; and
a hand skeletal joint position global optimization model, coupled with the joint 3D coordinate recognition model, and configured to optimize the hand joint position coordinate based on a finger bone length constraint and a finger bone joint constraint of the operator through a global optimization processing to improve the accuracy of the hand joint position coordinate, wherein the 2D joint recognition model, the joint 3D coordinate recognition model, and the hand skeletal joint position global optimization model are trained on a hand movement dataset by a first machine learning method; and
a movement analysis module, coupled with the recognition module, the movement analysis module comprising a hand movement analysis model configured to receive the hand joint position coordinate, analyze and compare the hand joint position coordinate of the operator with a standard hand movement parameter set, and generate an analysis and comparison result to evaluate the correctness of the at least one hand movement performed by the operator during the operation period, wherein the hand movement analysis model is trained on a standard operational hand movement dataset by a second machine learning method.

2. The system for hand movements recognition and analysis of claim 1, wherein the 3D coordinate is calculated by triangulating an intersection of a ray for each of the image capture modules, or by using a midpoint calculation of the shortest distance of each ray from the image capture modules as an approximate intersection point.

3. The system for hand movements recognition and analysis according to claim 1, wherein the movement analysis module further comprises:
a movement classification recognition model, configured to recognize and classify different types of hand movements based on the hand joint position coordinate in the continuous images captured by the image capture module through a third machine learning method.

4. The system for hand movements recognition and analysis according to claim 1, wherein the movement analysis module further comprises:
a movement and sequence accuracy recognition model, configured to analyze and compare the hand joint position coordinate with the standard hand movement parameter set, and determine whether the at least one hand movement of the operator and an operation sequence are correct.

5. The system for hand movements recognition and analysis of claim 1, further comprising:
a notification module, coupled with the movement analysis module, the notification module configured to send a notification message to alert the operator based on the analysis and comparison result generated by analyzing and comparing the hand joint position coordinate of the operator in the operational area during the operation period with the standard hand movement parameter set.

6. The system for hand movements recognition and analysis of claim 5, wherein the notification message further comprises an audio notification and a light signal notification.

7. The system for hand movements recognition and analysis of claim 6, wherein if the analysis and comparison result between the hand joint position coordinate of the operator in the operational area during the operation period and the standard hand movement parameter set does not match, the notification module generates the audio notification, wherein the audio notification is an error alert warning.

8. The system for hand movements recognition and analysis of claim 1, wherein the image capture module comprises a camera installed in the operational area.

9. The system for hand movements recognition and analysis of claim 1, wherein the operational area comprises an experimental operation platform, a sterile operation platform, and a cell handling station.

10. The system for hand movements recognition and analysis of claim 3, wherein the first machine learning method, the second machine learning method and the third machine learning method further comprise at least one of the following: an Artificial Neural Network (ANN), a Convolutional Neural Network (CNN), a Recurrent Neural Network (RNN), a Decision Tree, a Support Vector Machine (SVM), a Random Forest, a K-Nearest Neighbors (KNN) algorithm, K-Means Clustering, Principal Component Analysis (PCA), Linear Regression, Logistic Regression, Gradient Boosting Machines, a Deep Belief Network (DBN), a Recursive Neural Network (RecNN), Reinforcement Learning, an Autoencoder, Gaussian Processes, and a Complex Neural Network.

11. A method for hand movements recognition and analysis, for evaluating the correctness of at least one hand movement performed by an operator in an operational area during an operation period, the method comprising the following steps:
an image capture module capturing a continuous image of the at least one hand movement of the operator during the operation period;
a 2D joint recognition model of a recognition module recognizing a hand joint position of the operator based on the continuous image;
a joint 3D coordinate recognition model of the recognition module generating a hand joint position coordinate of the operator based on a plurality of continuous images from various perspectives captured by a plurality of image capture modules in the operational area during the operation period, wherein the hand joint position coordinate is a 3D coordinate;
a hand skeletal joint position global optimization model of the recognition module optimizing the hand joint position coordinate based on a finger bone length constraint and a finger bone joint constraint of the operator through a global optimization processing to improve the accuracy of the hand joint position coordinate, wherein the 2D joint recognition model, the joint 3D coordinate recognition model, and the hand skeletal joint position global optimization model are trained on a hand movement dataset by a first machine learning method; and
a hand movement analysis model of a movement analysis module receiving the hand joint position coordinate, analyzing and comparing the hand joint position coordinate of the operator with a standard hand movement parameter set, and generating an analysis and comparison result to evaluate the correctness of the at least one hand movement performed by the operator during the operation period, wherein the hand movement analysis model is trained on a standard operational hand movement dataset by a second machine learning method.

12. The method for hand movements recognition and analysis of claim 11, wherein the 3D coordinate is calculated by triangulating an intersection of a ray for each of the image capture modules, or by using a midpoint calculation of the shortest distance of each ray from the image capture modules as an approximate intersection point.

13. The method for hand movements recognition and analysis of claim 11, further comprising the following steps:
a movement classification recognition model of the movement analysis module recognizing and classifying different types of hand movements based on the hand joint position coordinate in the continuous images captured by the image capture module through a third machine learning method.

14. The method for hand movements recognition and analysis of claim 11, further comprising the following steps:
a movement and sequence accuracy recognition model of the movement analysis module analyzing and comparing the hand joint position coordinate with the standard hand movement parameter set, and determining whether the at least one hand movement of the operator and an operation sequence are correct.

15. The method for hand movements recognition and analysis of claim 11, further comprising the following steps:
a notification module sending a notification message to alert the operator based on the analysis and comparison result generated by analyzing and comparing the hand joint position coordinate of the operator in the operational area during the operation period with the standard hand movement parameter set.

16. The method for hand movements recognition and analysis of claim 15, wherein the notification message further comprises an audio notification and a light signal notification.

17. The method for hand movements recognition and analysis of claim 16, wherein if the analysis and comparison result between the hand joint position coordinate of the operator in the operational area during the operation period and the standard hand movement parameter set does not match, the notification module generates the audio notification, wherein the audio notification is an error alert warning.

18. The method for hand movements recognition and analysis of claim 11, wherein the image capture module comprises a camera installed in the operational area.

19. The method for hand movements recognition and analysis of claim 11, wherein the operational area comprises an experimental operation platform, a sterile operation platform, and a cell handling station.

20. The method for hand movements recognition and analysis of claim 13, wherein the first machine learning method, the second machine learning method and the third machine learning method further comprise at least one of the following: an Artificial Neural Network (ANN), a Convolutional Neural Network (CNN), a Recurrent Neural Network (RNN), a Decision Tree, a Support Vector Machine (SVM), a Random Forest, a K-Nearest Neighbors (KNN) algorithm, K-Means Clustering, Principal Component Analysis (PCA), Linear Regression, Logistic Regression, Gradient Boosting Machines, a Deep Belief Network (DBN), a Recursive Neural Network (RecNN), Reinforcement Learning, an Autoencoder, Gaussian Processes, and a Complex Neural Network.
